(19) United States
(12) Patent Application Publication
(10) Pub. No.: US 2005/0226920 A1
(43) Pub. Date: Oct. 13, 2005

(54) Method of decreasing nicotine withdrawal symptoms during smoking cessation.

(75) Inventor: Kirk George Voelker, Sarasota, FL (US)

Correspondence Address: Kirk G. Voelker, PO Box 25032, Sarasota, FL, 34239, US

(73) Assignee: Kirk George Voelker, Sarasota, FL (US)

(21) Appl. No.: 10/709,091

(22) Filed: Apr. 13, 2004

(51) Int. Cl. A61K 31/46; A61F 13/00; A61K 9/70

(52) U.S. Cl. 424/449; 514/304; 600/300; 705/2; 514/252.15

(57) ABSTRACT

Injecting a patient with a solution of anticolenergic medications can alleviate the symptoms of nicotine withdrawal. Used in conjunction with pre injection and post injection counseling, smoking cessation rates may be significantly improved.

### CROSS REFERENCE TO RELATED APPLICATIONS

U.S. Pat. No. 4,555,397 November, 1985 BACHYNSKY 514/557. U.S. Pat. No. 792,264 October, 1985 BOURNE 514/12. U.S. Pat. No. 4,621,074 November, 1986 BOURNE 514/12. U.S. Pat. No. 112,417 August, 1993 GUODONG 514/226. U.S. Pat. No. 213,111 March, 1994 CALLAWAY 424/464 U.S. Pat. No. 5,480,651 January, 1996 CALLAWAY 424/464. U.S. Pat. No. 803,723 February, 1997 VINER 514/291. U.S. Pat. No. 6,132,754 October, 2000 HUDSON424/423.

### BACKGROUND OF INVENTION

Cigarette smoking is the number one cause of preventable morbidity and mortality in the United States. Nicotine addiction is one of the most difficult addictions to overcome with recidivism equaling that of heroin addiction. The duality of nicotine addiction leads to both a physiologic addiction and a psychological addiction.

The physiologic principals of nicotine indicate that nicotine acts as an agonist at nicotinic receptors in the parasympathetic nervous system. The highest concentration of nicotine receptors resides centrally in the mid-brain. With chronic nicotine use there is a chronic block of nicotine receptors. In response to this chronic blockade of the nicotine receptors, there is a compensatory increase of acetylcholine through a process of enzyme induction of acetylcholine transferase. Therefore, when a smoker quits smoking, there is a withdrawal of this nicotine blockade nicotine and a marked increase of the neurotransmitter acetylcholine. This unchecked abundance of acetylcholine is in part responsible for the physiologic withdrawal of smoking cessation. Anticholenergic agents block the muscarinic receptors and attenuate the symptoms of nicotine withdrawal.

Glick, Jarvik and Nakauma have found that scopolamine decreased smoking patterns in monkeys. Bachynsky has demonstrated a significant improvement in smoking cessation rates of 500 smokers by using a solution of atropine and scopolamine potentiated by chlorpromazine injected subcutainiously and behind each ear.

Hyoscyamine is an anticholinergic agent with a which is soley responsible for the antimuscarinic action of atropine. Since the predominance of nicotinic receptors are located centrally at the mid-brain level, we have postulated that hyoscyamine is more effective than atropine. Clorpromazine has no anticholinergic properties however does have anxiolitic properties. Chlorpromazine also has a suboptimal side effect profile and is relatively contraindicated in patients taking popular serotonin uptake inhibitors. Hydroxyzine is a piperazine derivative antihistamine which has anticholinergic, antiemetic, antispasmodic and local anesthetic activity. We have had significant success in smoking cessation with an injection of hyoscyamine, scopolamine and clorpromazine and injection of hyoscyamine, scopolamine and hydroxyzine.

### SUMMARY OF INVENTION

By injecting a patient with an anticholinergic solution outlined by claim 1 and claim 2, excessive acetylcholine associated with nicotine withdrawal is blocked. This anticholinergic block decreases the effect of acetylcholine rebound bombardment of nicotine receptors in the midbrain thereby helping alleviate the withdrawal symptoms of smoking cessation. This injection is administered during a single office visit. The anticholinergic block is then continued via oral anticholinergic medications as outlined in claim 3 until the physiological effects of nicotine withdrawal are minimized. This method addresses the physiological aspects of nicotine withdrawal; however, there are also psychological associations linked to smoking cigarettes which must be overcome for successful smoking cessation. There are several methods of disassociating the habits of smoking by implementing a behavioral modification program and/or hypnosis. These associations may be addressed prior to the injection in order to enhance the success of a smoking cessation program. Unfortunately, many patients resume smoking well after the physiological addiction has resolved. Usually this is due to poor coping habits and lack of support. Therefore, smoking cessation success rates may also be enhanced by routine support and counseling after the physiologic withdrawal has resolved.

### DETAILED DESCRIPTION

Prior to the office visit, the patient completes a medical questionnaire and detailed smoking history questionnaire. If there is no contraindication to anticholinergic medications, an appointment is scheduled. Prior to the appointment, the patient reviews a smoking cessation video or other media and reads associated smoking cessation literature. There is a series of “homework” steps designed to disassociate the routine habits of smoking from the cigarette smoking process. During the office visit, a history and physical examination is performed and counseling may be done as well as any ancillary tests such as spirometry or electrocardiogram. Any patient with contraindications to the shot will be excluded from receiving the shot and alternative therapies such as hypnosis, will be offered. Patients felt suitable for the anticholinergic block method of smoking cessation will be injected with ½ of the amount of the solution outlined in claim 1 and claim 2. Patients are then allowed to sit in a darkened room and re examined after 5-15 min. Patients without excessive xerostomia and with normal pupillary constriction may then receive the remaining injected solution. The physician may choose to alter the components of the remaining shot depending upon findings on the physical exam. The patient is then observed in the office until stable for discharge. The patient is instructed not to drive or drink alcohol within 8 hours of the injection. The next morning, the patient is started on anticholinergic medications such as scopolamine patches, belladona tablets, hydroxyzine tablets, probanthene tablets, to continue the anticholinergic block for up to 14 days.

Though this anticholinergic block may be successful without an further follow up, many patients need to conquer the psychological addiction of smoking. To address this psychological addiction numerous methods of support such as telephone counseling, internet discussion boards, internet chat rooms, group telephone conference calls and group meetings may be implemented.

1. A method of minimizing nicotine withdrawal symptoms by injecting a patient with an anticholinergic medication consisting of from 0.0 to 0.8 mg of hyoscyamine, 0.0 to 0.8 mg scopolamine and 0 to 100 mg of hydroxyzine in a suitable carrier.

2. A method of minimizing nicotine withdrawal symptoms by injecting a patient with an anticholinergic medication consisting of from 0.0 to 0.8 mg of hyoscyamine, 0.0 to 0.8 mg scopolamine and 0 to 50 mg of chlorpromazine in a suitable carrier.

3. A method of minimizing nicotine withdrawal symptoms by injecting a patient with an anticholinergic medication consisting of from 0.0 to 0.6 mg of atropine, 0.0 to 0.8 mg scopolamine and 0 to 100 mg of hydroxyzine in a suitable carrier.

4. A method of minimizing nicotine withdrawal symptoms by injecting a patient with an anticholinergic medication consisting of from 0.0 to 0.4 mg of glycopyrrolate, 0.0 to 0.8 mg scopolamine and 0 to 100 mg of hydroxyzine in a suitable carrier.

5. A method of continuing the effects of the anticholinergic shot described in claim 1, claim 2, claim 3 and claim 4 by concomitantly prescribing any combination of scopolamine patches, scopolamine tablets, belladona tablets, hydroxyzine tablets, probanthene tablets using accepted pharmacological principals.

6. A method of decreasing anxiety associated with nicotine withdrawal after the anticholinergic shot described in claim 1, claim 2, claim 3 and claim 4 by utilizing hydroxyzine, benzodiazepines or clonidine.

7. A method of disassociating the habits of smoking by implementing a behavioral modification program and/or hypnosis via individual or group counseling, video, CD, DVD, streaming video or audiotape prior to, or after receiving the anticholinergic shot described in claim 1, claim 2, claim 3 and claim 4.

8. A method of helping reduce the psychological dependence on nicotine by counseling after administering the anticholinergic shot described in claim 1, claim 2, claim 3 and claim 4, utilizing telephone conference calls, internet discussion boards, internet chat rooms, group meetings and/or individualized telephone follow up.

9. A method of helping reduce the physiological and psychological dependence on nicotine by using antidepressants such as serotonin reuptake inhibitors, bupropion, or buspirone prior to or in conjunction with receiving the anticholinergic shot outlined in claim 1, claim 2, claim 3 and claim 4.
